(19) United States
(12) Patent Application Publication
(10) Pub. No.: US 2025/0230415 A1
(43) Pub. Date: Jul. 17, 2025

(54) COMPOSITIONS AND METHODS FOR IMPROVING TREATMENT OF CANCER

(71) Applicant: Cypre Inc., San Fransisco, CA (US)

(72) Inventors: Kolin Hribar, San Fransisco, CA (US); Bin Xue, El Cerrito, CA (US)

(21) Appl. No.: 18/578,676

(22) PCT Filed: Jul. 15, 2022
(86) PCT No.: PCT/US2022/037373

§ 371 Date: Jan. 11, 2024

Related U.S. Application Data
(60) Provisional application No. 63/222,150, filed on Jul. 15, 2021.

(51) Int. Cl. C12N 5/09; A61K 40/11; A61K 40/42; A61K 40/50; C12N 5/0783; G01N 33/50

(52) U.S. Cl. CPC C12N 5/0693; A61K 40/11; A61K 40/428; A61K 40/50; C12N 5/0638; G01N 33/5085; C12N 2502/1114; C12N 2502/1323; C12N 2513/00; C12N 2533/90; G01N 2800/52

(57) ABSTRACT

Systems, compositions, and methods are described that utilize three dimensional tumor models incorporating tissue barrier (such as a stromal barrier) around tumor cells in culture, which permit replication in vivo immune cell response to tumor cells. Such tumor models are used to accurately assess appropriate therapeutic modes and protocols that are likely to be effective against the individual's tumor. Such tumor models can be used to selectively expand immune cells that are responsive to tumor cells utilized in the model. Such an expanded population of immune cells can subsequently be utilized therapeutically.

This application claims the benefit of U.S. Provisional Patent Application No. 63/222,150 filed on Jul. 15, 2021. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is cancer drug discovery and personalized medicine, specifically personalized treatment of cancer.

### BACKGROUND

The following background description includes information that may be useful in understanding the present invention. It is not an admission that any of the information provided herein is prior art or relevant to the presently claimed invention, or that any publication specifically or implicitly referenced is prior art.

Cancer is a highly diverse condition, with neoplastic cells arising from a variety of tissues and cell types, a wide range of presentations (e.g., solid, non-solid), and phenotypes (vascular, non-vascular, non-malignant, malignant, localized, metastatic, chemosensitivity, etc.). In addition, individual immune responses to cancer can vary widely (e.g., inflamed, excluded, cold, “immune desert”). Clinicians have a wide variety of therapeutic approaches at their disposal, including chemotherapy, radiotherapy, hyperthermia, antibody-based immunotherapy, and cell-based immunotherapy, however they lack adequate tools to determine which therapeutic approach is most likely to be effective for a specific individual. Additionally, current tools utilized by drug discovery and preclinical researchers have very limited capabilities for recreating the complex tumor microenvironment in vitro for advancing lead candidate compounds and therapies to the clinic.

United States Patent Application Publication No. 2012/0208706, to Downing et al., and United States Patent Application Publication No. 2018/0363066, to Chalmers et al., for example, provide methods for rapidly identifying specific mutations in tumor cells. While such mutations may provide a degree of therapeutic insight, being limited to genetic information they do not provide direct data related to effective therapy of such a tumor in situ. All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

United States Patent Application Publication No. 2018/0252703 describes methods for determining tumor sensitivity by exposing three-dimensional cell cultures that include neoplastic cells to an anti-cancer agent. Similarly, United States Patent Application Publication No. 2019/0029235, to Sikora and Pathak, describe test systems based on xenografts of tumor cells onto chick chorioallantoic membranes. Such approaches, however, fails to reproduce tumor architecture, tissues that may surround a patient's tumor, and the patient's own immune response to the tumor. As such they do not provide data that is directly useful for determining a therapeutic approach.

United States Patent Application Publication No. 2019/309264, to Presnell et al., the use of three dimensional cancer models that include tumor cells that are inserted into a bioprinted stromal microenvironment to determine the effect of candidate therapies on various aspects of tissue growth, including their application to individualized therapy. The described method, however, fails to secure the implanted tumor cells, which complicates visualization. In addition, the described methods fail to account for pathology findings related to the tumor. Finally, relying on deposition to provide the stromal microenvironment necessarily limits the dimensions that can be achieved.

Thus, there is still a need for systems and methods that can provide a tumor model adequate for accurate prediction of the success of a therapeutic method.

### SUMMARY OF THE INVENTION

The inventive subject matter provides systems, compositions, and methods that provide planar three dimensional constructs incorporating cells characteristic of cancer (e.g., tumor cells, fibroblasts, etc.) and other disease states, as well as therapeutic compounds and/or immune cells in order to facilitate treatment of cancer and other disease states. Such constructs can also be used for selective clonal expansion of immune cells, which in turn can be utilized therapeutically.

Embodiments of the inventive concept include methods for diagnosing and predicting treatment for a cancer patient by obtaining data related to a pathology of a tumor of the cancer patient, determining a distribution of cancer cells, stromal cells, or immune cells within or proximal to the tumor, generating a plurality of three dimensional models of the tumor including two or more of (i) tumor cells obtained from the cancer patient, (ii) stromal cells, and/or (iii) immune cells. Cancer cells, immune cells, and/or stromal cells can be obtained from a cancer patient or from a non-patient source (i.e., an allogenic source, tissue or cell culture, explanted tissue, etc.). In such methods each of the three dimensional models reflects a distribution of cancer cells, stromal cells, or immune cells as indicated by the pathology, each of the plurality of three dimensional models is deposited on a test surface, and tumor cells of each of the three dimensional models are coupled to the test surface or an acellular layer coupled to the test surface. The plurality of three dimensional models are exposed to a plurality of anti-cancer treatments, and the effects of the anti-cancer treatments on the three dimensional tumor models are characterized. Therapeutic efficacy in the cancer patient of one or more of the anti-cancer treatments can be predicted based on their effects on the three dimensional tumor models.

Each of the plurality of three dimensional tumor models can include a first compartment and a second compartment, the first compartment having a first face and a second face, where the first face is in contact with the test surface and the second face is in contact with the second compartment, and where one or both of the first compartment and the second compartment can (optionally) include one or more of extracellular matrix, biomaterial, and a biopolymer scaffold. Tumor cells and immune cells can be co-located in this first compartment to provide a model of an immune inflamed tumor. Alternatively, tumor cells can present in and immune cells absent from the first compartment, with immune cells present in the second compartment or on a surface of the second compartment to provide a model of an immune excluded tumor. In some embodiments stromal cells are positioned proximal to the first compartment, such that stromal cells (e.g., fibroblasts, endothelial cells, mesenchymal stem cells (MSC), adipocytes, and/or pericytes) are interposed between tumor cells and immune cells (e.g., stimulatory immune cells of the innate or acquired immune system, suppressive immune cells of the innate or acquired immune system, peripheral blood mononuclear cells (PBMCs), T cells, NK cells, B cells, dendritic cells, mast cells, neutrophils, and/or macrophages). Alternatively, in some embodiments tumor cells and stromal cells can co-located in at least a portion of the plurality of three dimensional tumor models. In some embodiments tumor cells and immune cells are co-located in at least a portion of the plurality of three dimensional tumor models. In some embodiments immune cells and stromal cells are co-located in at least a portion of the plurality of three dimensional tumor models. In some embodiments tumor cells, stromal cells, and immune cells are co-located in at least a portion of the plurality of three dimensional tumor models. In some embodiments, immune cells are absent from the tumor models in order to provide a model of an immune desert tumor.

Such methods can include provision of a liquid media to a portion of the three dimensional models. The liquid media can include an immune cell and, optionally, a stromal cell. Tumor cells, stromal cells, and/or immune cells are obtained from a surgical sample of the tumor, biopsy from the tumor, normal tissue from the cancer patient, obtained from a circulating fluid of the cancer patient, or can be obtained from established cell lines. In some embodiments the immune cells are activated T cells that are prevented from infiltrating a three dimensional tumor that is under controlled conditions without treatment. Alternatively, the immune cells can activated T cells that are able to infiltrate a three dimensional tumor that is under a controlled conditions without treatment.

Data utilized in such methods can be obtained by one or more of immunohistochemistry (IHC), flow cytometry, gene expression, and methods informative of tumor microenvironment or makeup of the individual patient's tumor. IHC data so provided can be related to relative orientation and location of tumor, immune, and stromal compartments of the cancer patient. Such data can be collected by automated analysis, such as high content imaging, cell sorting, flow cytometry, proteomic analysis, expression analysis, and/or genome sequencing. In some embodiments data collected by automated analysis can be assessed to quantitate one or more of immune cell infiltration, tumor cell death, and other tumor microenvironment changes within the plurality of three dimensional tumor models

Anti-cancer treatments evaluated in such methods can be a cancer targeted therapy, an immunomodulator, a chemotherapy, a repurposed drug conventionally utilized for treatment of a condition other than cancer, radiation, or a combination of two or more of these. Three dimensional tumor models utilized in such methods can be generated using a liquid handling system, such as a bioprinter, and can be automated. Such a liquid handling system can include a photomask and a light source. Such a liquid handling system can deposit one or more of an extracellular matrix, biomaterial, or biopolymer scaffold onto the test surface.

Some embodiments of the inventive concept are methods of optimizing cancer treatment for a cancer patient by generating a first data set that includes historical predicted therapeutic efficacies developed using a method as described above for a plurality of historical cancer patients, generating a second data set that includes therapeutic outcomes for the plurality of historical cancer patients, and providing an artificial intelligence system with a learning algorithm configured to access the first and second data sets to generate a proposed treatment plan algorithm that correlates or otherwise associates predicted therapeutic efficacies and therapeutic outcomes, and applying the treatment plan algorithm to the predicted therapeutic efficacies to provide or propose one or more treatment protocols likely to be effective for treatment of the cancer patient. Such an artificial intelligence system can be configured as a neural network. In some embodiments the artificial intelligence system is accessed via an information network, and can be accessed via a subscription service.

Some embodiments of the inventive concept are methods of optimizing cancer treatment for a cancer patient by generating a first data set comprising historical predicted therapeutic efficacies using a method as described above for a plurality of historical cancer patients, generating a second data set that includes therapeutic outcomes recorded for the plurality of historical cancer patients, and providing an artificial intelligence system comprising a learning algorithm with the first and second data sets to generate a treatment plan algorithm correlating predicted therapeutic efficacies and therapeutic outcomes, and applying the treatment plan algorithm to data related to a pathology of a tumor of the cancer patient to report or propose a treatment plan for the cancer patient. Such an artificial intelligence system can be configured as a neural network. Such an artificial intelligence system can be configured as a neural network. In some embodiments the artificial intelligence system is accessed via an information network, and can be accessed via a subscription service.

Embodiments of the inventive concept include methods for performing a three-dimensional cell-based assay by obtaining a planar, three dimensional construct that includes a planar, or essentially planar, first layer of extracellular matrix or hydrogel in a culture vessel, where a first cell (e.g., immune cell, a target cells, or a combination of immune and target cells) is provided below or mixed within the first layer and a second cell (e.g., an immune cell, a target cell, and a combination of immune and target cells) is provided above the first layer, and measuring or otherwise characterizing an interaction between the first cell and the second cell interaction over a period of time. The second cell can be a tumor cell, a non-tumor diseased mammalian cell, a, pathogen, a bacteria, a virus; a source of chemokine, a source of chemoattractant, a source of a stimulus that enables an immune response, and/or an immune cell (e.g., a T cell, a B cell, a dendritic cell, a macrophage, an, NK cell, a peripheral blood mononuclear cells (PBMC), and/or derivatives of same). The second cell can be of human origin, non-human animal origin, or non-animal origin. Such a construct can further include a stromal cell, a fibroblast, an endothelial cell, an immune cell, a normal tissue cell, a diseased cell, a pathogen, a bacteria, and/or a virus.

Such a construct can include a biomaterial, such as a polymer of natural or synthetic origin; a decellularized tissue, a protein, a dextran, alginate, a poly(ethylene glycol), a collagen, a gelatin, hyaluronic acid, combinations thereof, and/or chemically modified derivatives thereof formulated for chemical or physical crosslinking. Such constructs can include at least one layer that includes a polymer that has been polymerized using electromagnetic radiation, temperature, and/or time. Such a construct can include a second layer that is planar, or essentially planar. At least one layer can be selected to be suitable for biocompatibility, elastic modulus, porosity, and degradability to enable nutrient exchange and cell interactions within a layer of a construct or between layers of a construct.

Suitable constructs for these methods can be produced using standard or custom molding methods, photolithography, bioprinting, and/or techniques that provides control of size, shape and planarity of one or more layers of the construct The first layer of such constructs can have a defined thickness and between 10 micrometers and 2 millimeters, preferably between 100 micrometers and 1 millimeter, and can have a stiffness of from 50 Pa to 20 kPa.

Embodiments of the inventive concept include a three-dimensional cell-based assay system that includes a first planar, or essentially planar, layer of extracellular matrix or hydrogel in a culture vessel, a first cell (e.g., immune cell, a target cells, or a combination of immune and target cells) below, mixed within, or above the first layer, a second layer of extracellular matrix or hydrogel on top of the first layer, and a second cell (e.g., immune cell, a target cell, a combination of immune and target cells, a tumor cell, a non-tumor diseased mammalian cell, a, pathogen, a bacteria, a virus; a source of chemokine, a source of chemoattractant, and a source of a stimulus that enables an immune response) mixed in the second layer or located on top of the second layer. Such second cells can be of human origin, non-human animal origin, or non-animal origin. Suitable immune cells include Such an assay system can further include a stromal cell, a fibroblast, an endothelial cell, an immune cell, a normal tissue cell, a diseased cell, a pathogen, a bacteria, and/or a virus. Such an assay system can include a second layer that is planar, or essentially planar. Such an assay system can include an immune cell, such as a T cell, a B cell, a dendritic cell, a macrophage, an, NK cell, a peripheral blood mononuclear cells (PBMC), and/or derivatives of these.

Such assay systems can include a biomaterial, such as a polymer of natural or synthetic origin; a decellularized tissue, a protein, a dextran, an alginate, a poly(ethylene glycol), a collagen, a gelatin, hyaluronic acid, combinations thereof, and chemically modified derivatives thereof formulated for chemical or physical crosslinking Such an assay system can include at least one layer that includes a polymer produced by polymerization using electromagnetic radiation, temperature, or time.

At least one layer of such an assay system is selected to be suitable for biocompatibility, elastic modulus, porosity, and degradability to enable nutrient exchange and cell interactions within a layer or between a plurality of layers. Such an assay system can be fabricated using standard or custom molding, photolithography, bioprinting, or a technique that provides control of size, shape and planarity of a layer of the construct. The first layer of such an assay system can have a defined thickness and between 10 micrometers and 2 millimeters, preferably between 100 micrometers and 1 millimeter, and can have a stiffness of from 50 Pa to 20 kPa.

Embodiments of the inventive concept include methods of assessing efficacy of a therapeutic modality by measuring at least one of cell viability, cell conformation, and presence of cell apoptosis markers in either of a first or second cell in a method as described above, Such measurements can be obtained by at least one of live cell image analysis, immunofluorescence, flow cytometry, proteomic analysis, and genomic analysis. Such methods can include obtaining measurements of immune cell infiltration into a layer of the construct, immune cell infiltration or into an aggregate of target cells, analysis of protein secretions, characterization of cytokines, observable changes to target cells or immune cells, characterization of apoptosis in target cells or immune cells, characterization of proliferation in target cells or immune cells, characterization of cell aggregation size in target cells or immune cells, characterization of a phenotypic change in a target cells or immune cells, and/or characterization of genotypic changes in target and immune cells. Such methods can include subjecting at least a portion of a layer of a three dimensional construct or assay system to at least one of chemical digestion, enzymatic digestion, mechanical digestion, or disruption by addition of a chelating agent. Such steps can include robotic selection or aspiration to recover a cell released by digestion or disruption of the layer.

Embodiments of the inventive concept include methods for providing individualized medicine by performing a method as described above, where the method incorporates a patient or pathogen-derived target cell and an autologous or allogenic immune cell, subjecting the construct or assay system used to one or more treatment modalities (e.g., radiotherapy, chemotherapy, targeted therapy, and/or an immunotherapy); determining efficacy of the one or more treatment modality within the construct or assay system, and providing efficacy information based on such determinations to a medical professional to assist in clinical decision-making for a specific patient or a group of patients.

Embodiments of the inventive concept include methods of expanding an immune cell population by performing a method of as described above where the three dimensional construct or assay system include a immune cells cultured proximal to a target cell within a first or second layer, providing an incubation period sufficient to allow immune cells to proliferate to generate an expanded immune cell population, and recovering the expanded immune cell population from the three dimensional construct or assay system. The expanded immune cell population is modified following recovery, for example by genetic modification, additional expansion, or a combination thereof. Such immune cells can be of autologous or allogeneic origin.

Embodiments of the inventive concept include methods of producing an antibody, by performing a method of clonal expansion as described above where the three dimensional construct or assay system includes an immune cell that is a cultured B cell or a hybridoma cell to generate a population of expanded immune cells, selecting a cell subpopulations from the population of expanded immune cells; and collecting the antibody from the selected cell subpopulation. Such selection can be performed using live imaging, manual picking, and/or robotic picking.

Embodiments of the inventive concept include methods of endogenous cell therapy by performing a method of clonal expansion as described above, where the cell to be expanded is an immune cell derived from peripheral blood, a spleen, or a disease microenvironment, expanding the immune cell to generate an expanded population of immune cells, and infusing at least a portion of the expanded population of immune cells into an individual in need of treatment. In some embodiments a subpopulation of immune cells (which can be assessed for efficacy in the patient) is separated from the expanded population of immune cells and infused into the patient in need of treatment. Assessment of patient efficacy can be performed after infusion or prior to infusion.

Embodiments of the inventive concept include methods of providing an immune cell having a specified phenotype (e.g., peripheral blood monocyte or a tumor infiltrating lymphocyte), by generating a three dimensional culture that includes an antigen producing cell (e.g. a tumor cell or a pathogen cell) layer, contacting the three dimensional culture with an immune cell to generate a coculture, and incubating the coculture for a period of time sufficient to generate the immune cell having the specified phenotype, incubating the coculture for a period of time sufficient to generate the immune cell having the specified phenotype. Suitable phenotypes include a cytolytic effector T cell, an NK cell, a macrophage, a memory T cell, such as a stem cell memory T cell, a central memory T cell, an effector memory T cell, and a transitional memory T cell. The period of time can be select to result in a stimulatory effect on the immune cell, such as antigen-specific stimulation, CD3/CD28 stimulation, CD3/CD28 stimulation with CD137, and co-stimulation by one or more cytokines (e.g., IL2, IL7, and IL15), Such a three dimensional culture can include a stromal cell layer (which can include fibroblasts) interposed between the antigen producing cell layer and the immune cell. In some embodiments the three dimensional culture can include a cytokine-producing cell (e.g., stromal cells, a fibroblast, an endothelial cell, a cytokine-producing immune cell, and/or a dendritic cell).

Such a three dimensional culture can include a hydrogel or an extracellular matrix component. The three dimensional culture can include a dextran, a gelatin, a collagen, a hyaluronic acid, and a polyethylene glycol, and at least a portion of the three dimensional culture can be degradable by a metal metalloproteinase. The three dimensional culture can have a thickness of from 20 μm to 2 mm, and can have a stiffness of from 50 Pa to 20 kPa. The three dimensional culture can be patterned into distinct areas and/or orientations, for example within a vessel or well or on a surface. In some embodiments the three dimensional culture can include a porous membrane.

Embodiments of the inventive concept include methods of providing an immunotherapy, by isolating an immune cell have a specified phenotype produced by a method as described above to provide an isolated immune cell. contacting the isolated immune cell with a pharmaceutically acceptable carrier to generate an immunotherapeutic composition, and administering the immunotherapeutic composition to an individual in need of treatment (e.g., by infusion). Suitable pharmaceutically acceptable carriers include a liquid medium that is suitable for injection or infusion. In some embodiments the pharmaceutically acceptable carrier is a biocompatible hydrogel or tissue scaffold, which can be administered by local application to a portion of the individual to be treated.

Embodiments of the inventive concept include systems for providing an immune cell having a specified phenotype, which include a three dimensional culture that include an antigen producing cell layer (e.g., a pathogenic cell layer, a tumor cell layer, etc.) and an immune cell (e.g., a peripheral blood monocyte or a tumor infiltrating lymphocyte) in coculture within the three dimensional culture, where the three dimensional culture is configured to enhance clonal expansion of the immune cell having the specified phenotype. The three dimensional culture of such a system can include a stromal cell layer that is interposed between the antigen producing cell or tumor cell layer and the immune cell. The three dimensional culture can have a defined thickness of between 10 micrometers and 2 millimeters, preferably between 100 micrometers and 1 millimeter, and can have a stiffness of from 50 Pa to 20 kPa. In such a system the three dimensional culture can patterned into distinct areas and/or orientations.

In such systems the three dimensional culture can include a stromal cell layer interposed between the antigen producing cell or tumor cell layer and the immune cell. Such a stromal cell layer can include fibroblasts. In some embodiments the three dimensional culture includes a hydrogel and/or an extracellular matrix component. At least a portion of the three dimensional culture is degradable by a metal metalloproteinase. In some embodiments the three dimensional culture can include a dextran, a gelatin, a collagen, a hyaluronic acid, an alginate, a polyethylene glycol, and/or a porous membrane.

In such systems the three dimensional culture can be configured to provide a stimulatory effect on the immune cell, such as antigen-specific stimulation, CD3/CD28 stimulation, CD3/CD28 stimulation with CD137, and co-stimulation by one or more cytokines (e.g., IL2, IL7, and/or IL15). In some embodiments the three dimensional culture includes a cytokine-producing cell. Suitable cytokine producing cells include a fibroblast, an endothelial cell, a cytokine-producing immune cell, and a dendritic cell.

In such systems the immune cell having a selected phenotype can be a memory T cell. Such a memory T cell can be a stem cell memory T cell, a central memory T cell, an effector memory T cell, and a transitional memory T cell. In some embodiments the immune cell having a selected phenotype is selected from the group consisting of one or more of a cytolytic effector T cell, an NK cell, and a macrophage.

Embodiments of the inventive concept include use of an immune cell having a specified phenotype to manufacture an immunotherapeutic composition, which includes isolating an immune cell have a specified phenotype produced by a method as described above and providing the immune cell having the specified phenotype in a pharmaceutically acceptable carrier. Such a pharmaceutically acceptable carrier can be a liquid medium suitable for infusion. Alternatively, the pharmaceutically acceptable carrier can be a biocompatible hydrogel or tissue scaffold.

Embodiments of the inventive concept include a three-dimensional cell-based assay system that has a first planar, or essentially planar, layer (which can be a product of a standard or custom molding, a photolithography, or bioprinting) of extracellular matrix or hydrogel in a culture vessel, a first cell (e.g., an immune cell, a target cell, or a combination of immune and target cells) below or mixed within the first layer, and a second layer that includes a second cell (e.g., an immune cell, a target cell, a combination of immune and target cells, a tumor cell, a non-tumor diseased mammalian cell, a, pathogen, a bacteria, a virus a source of chemokine, a source of chemoattractant, and/or a source of a stimulus that enables an immune response), where the second layer is planar. Such second cells can be of human origin, non-human animal origin, or non-animal origin. Such a system can include one or more other cell types, such as a stromal cell, a fibroblast, an endothelial cell, an immune cell, a normal tissue cell, a diseased cell, a pathogen, a bacteria, a virus, a T cell, a B cell, a dendritic cell, a macrophage, an, NK cell, a peripheral blood mononuclear cells (PBMC), and derivatives of same. In some embodiments the second layer can include extracellular matrix or hydrogel. Such systems can include a biomaterial or biocompatible material, such as a polymer of natural or synthetic origin; a decellularized tissue, a protein, a dextran, an alginate, a poly(ethylene glycol), a collagen, a gelatin, hyaluronic acid, combinations thereof, and chemically modified derivatives thereof formulated for chemical or physical crosslinking. At least one layer of such a system can include a polymer polymerized using electromagnetic radiation, temperature, or time. At least one layer of such a system can be selected to be suitable for biocompatibility, elastic modulus, porosity, and degradability to enable nutrient exchange and cell interactions within a layer or between layers In such systems the first layer can have a defined thickness and between 10 micrometers and 2 millimeters, preferably between 100 micrometers and 1 millimeter.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 schematically depicts an exemplary planar three dimensional construct of the inventive concept.

FIG. 2 schematically depicts movement of cells within a planar three dimensional construct of the inventive concept.

FIGS. 3A to 3D provide a photomicrograph of infiltrating immune cells and effects of an immune checkpoint inhibitor on infiltration of such cells and subsequent effects on tumor cells. FIG. 3A provides a photomicrograph of infiltration of immune cells through an extracellular matrix (ECM) barrier placed between a layer containing tumor cells and a layer containing immune cells in a planar three dimensional construct of the inventive concept. FIG. 3B provides a histogram of typical data illustrating the effects of application of an immune checkpoint inhibitor on immune cell infiltration in such a construct. FIG. 3C provides a histogram of typical data illustrating the effects of the immune checkpoint inhibitor on tumor cell death in such a construct. FIG. 3D shows typical data illustrating the effects of a checkpoint inhibitor on tumor area in such a construct.

FIG. 4 provides a schematic depiction and photomicrographs of clonal expansion of immune cells using a planar three dimensional construct of the inventive concept. Clonal expansion is apparent on inclusion of tumor cells in the underlying layer.

FIG. 5 depicts typical results for studies directed to effects of fibroblasts on tumor sensitivity to drug therapy. As shown, presence of fibroblasts in a layer overlying tumor cells can provide a protective effect.

FIG. 6 depicts typical results of studies of immune cell distribution in tumor cell-containing planar three dimensional constructs that omit or include an intervening layer containing fibroblasts. The left panel shows a photomicrograph of such a construct without such an intervening fibroblast layer, in which immune cells are randomly distributed. The right panel shows a photomicrograph of such a construct that includes an intervening layer containing fibroblasts, in which tumor cells are nonrandomly distributed along the fibroblast spindles.

FIG. 7 provides photomicrographs through a planar three dimensional construct of the inventive concept, in which tumor cells are cultured with a fibroblast layer, in the presence of immune cells (e.g., PBMCs).

FIG. 8 provides photomicrographs through a planar three dimensional construct of the inventive concept, in which tumor cells are cultured with a fibroblast layer, in the absence of immune cells.

FIG. 9 provides photomicrographs showing significant aggregation of immune cells (PBMCs) along a fibroblast network that overlies tumor cells within a planar three dimensional construct of the inventive concept.

FIG. 10 shows typical results of flow cytometry studies performed to characterize immune cells cultured in a planar three dimensional construct of the inventive concept that incorporates cultured tumor cells.

FIG. 11 shows typical results of flow cytometry studies performed to characterize immune cells cultured in a planar three dimensional construct of the inventive concept that incorporates tumor cells taken from a patient.

FIG. 12 shows typical results of flow cytometry studies demonstrating selective enhancement of clonal expansion of immune cells cultured in a planar three dimensional construct of the inventive concept that incorporates tumor cells.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

The inventive subject matter provides apparatus, systems and methods that provide a three dimensional tumor model that can incorporate an analog of a tissue barrier (such as a stromal barrier) around tumor cells in culture, and can replicate the individual's immune cell response to tumor cells. Such a tumor model can be used to accurately assess appropriate therapeutic modes and protocols that are likely to be effective against the individual's tumor. In some embodiments such tumor models can be used to selectively expand immune cells responsive to tumor cells utilized in the model. Such an expanded population of immune cells can subsequently be utilized therapeutically (e.g., by returning at least a portion of the expanded immune cell population to the individual with the tumor).

Various objects, features, aspects and advantages of the inventive subject matter will become more apparent from the following detailed description of preferred embodiments, along with the accompanying drawing figures in which like numerals represent like components.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

One should appreciate that the disclosed techniques provide many advantageous technical effects including accurate and effective treatment of cancer.

The term “essentially planar” as used within this application refers to a feature surface that deviates 10% or less from planarity.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Central to embodiments of the inventive concept is the generation of a three dimensional tissue model. Any suitable method for doing so may be used. In preferred embodiments the apparatus and methods described in U.S. Pat. Nos. 10,073,346, 10,423,071, and U.S. patent application Ser. No. 16/156,663, which represent work by the inventors and are hereby incorporated by reference. These describe methods in which cells are introduced in a liquid suspension that includes one or more photoactivatable polymer precursor(s). Exposure to a suitable wavelength of light through a photomask having a specified configuration results in polymerization and formation of a three dimensional solid a desired profile (as provided by the photomask) and in which cells are suspended. The dimensions of the three dimensional solid are a function of a number of factors, including the area illuminated, the volume of cell suspension provided, and amount of light provided (via intensity, exposure, etc.), presence and configuration of a pillar inserted into the culture plate well, etc.

Such methods permit generation of cell-containing solids in a wide variety of shapes and sizes. Repeated cycles of solid generation provide the creation of a wide variety of complex geometries resulting from the sequential use of differently configured photomasks and subsequent generation of overlying three dimensional solids (which can incorporate suspended cells, growth factors, chemotactic factors, etc.). These methods also permit the generation of a wide variety of complex tissue models through the incorporation of different cell types into different portions of the resulting three dimensional solids.

Embodiments of the inventive concept can be directed to a wide variety of disease conditions. While applications to cancer (e.g., through the use of tumor models) is described below, Inventors contemplate application of such cell-based models to non-tumor disease states. Other non-tumor disease models may include fibrosis, inflammation, CNS diseases, and others which obviate the utility of mutated normal cells which behave in a dysfunctional phenotypic or genotypic manner. Said models may be supplemented with microenvironment cells in a similar fashion as the tumor models-stromal cells (e.g., fibroblasts) and immune cells (T cells, B cells, NK cells, macrophages, monocytes, etc.).

In some embodiments, non-diseased cell models may be developed in a similar fashion to function as toxicity assay for the compounds tested with the disease (tumor) model; said models may comprise patient-derived or allogenic donor cells, or cell lines of the specific healthy tissue lineage—e.g., heart, neural (brain, CNS), skin, bone, etc. Moreover, the healthy cells may be derived from stem cells, such as induced pluripotent stem cells (iPSCs). The healthy 3D models may be in static or dynamic fluid-flow culture, separate or connected to the disease (tumor) model, to simulate a human-like closed system of drug compound testing.

It should be appreciated that systems of the inventive concept can, in addition to including planar three dimensional constructs as described below, include mechanisms to support methods performed that utilize such constructs. Such components include instruments for liquid handling and dispensing (e.g., pipettors), positioned to dispense liquids (e.g., cells in suspension, drugs in solution, etc.) into vessels containing such constructs and/or to collect liquids from such vessels (e.g., to collect expanded clonal cell, etc.). Such components can include sensor instruments for observing or otherwise monitoring planar three dimensional constructs, such as suitable optical devices (e.g., cameras, microscopes, etc.). Similarly, such systems can include control systems that are communicatively coupled to such liquid handling and sensor instrument devices. Such control systems can permit manual control via a suitable interface, and/or can include a computational device for performance of user-specified tasks. Such user-specified tasks can include maintenance and/or monitoring of the planar three dimensional construct, and can include functional tasks utilizing the planar three dimensional construct. Such functional tasks can include addition of specified drugs (e.g., for screening studies), dispensing of immune cells for clonal expansion, collection of clonally expanded cells, etc. as described below. Such a controller can include a memory device that encodes instructions for performance of such tasks.

Such a controller can be co-located with other system components, or can be located remotely. Such a remote server can be accessed via a server. Throughout the following discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

One embodiment of the inventive concept is a method of predicting an effective treatment for an individual patient. In an initial step features and properties of the individual patient's tumor are characterized. This can be accomplished by any suitable method, including genome, expressome, and/or proteome analysis of samples taken from the tumor and/or circulating cancer cells (e.g., identification of specific surface markers via immunohistochemistry, flow cytometry, immunoassay, etc.), and/or imaging. Such characterization can be used to determine the presence or absence of cells of the immune system, and permit classification of the tumor as immune inflamed (showing evidence of inflammation and/or presence of immune cells within the tumor), immune excluded (e.g., immune cells are present but are stopped at a stromal cell barrier surrounding the tumor), or an immune “desert” (i.e., no immune cells apparent).

Following characterization of the tumor and its immune status a three dimensional model of the individual patient's tumor as it is present within the patient's normal tissue can be prepared. As noted above this model can be prepared by any suitable method. This three dimensional model is deposited on a suitable solid surface, such as the floor of a test well or the surface of a membrane. In such a model the naturally occurring tissue layers can be represented by layers of deposited cells and/or one or more polymer matrix(ces) including such cells. Such a polymer matrix can include supportive components such as components of the extracellular matrix, biomaterials, biopolymer scaffolding, photopolymer precursors, etc.,

Tumor cells can be obtained from a patient, or can be obtained in the form of a tumor cell line. Such a tumor cell line can be representational of a cell of a patient's tumor, for example in terms of tissue of origin, tumor type, and/or expression of tumor markers. In a preferred embodiment tumor cells are obtained from the individual patient, for example through surgery, biopsy, and or collection of circulating tumor cells. In some embodiments tumor cells can be provided as cells or clumps of cells held in suspension. In other embodiments tumor cells can be provided as one or more intact portions of the tumor mass. In other embodiments, said tumor cells or tumor masses can be passaged in an animal model (e.g. mouse to generate a patient-derived xenograft (PDX) mode, from which the cells may then be excised for in vitro cell culture studies. In even further embodiments, cells may be expanded and established in cell culture to allow for expansion potential (either in perpetuity as an “immortalized” cell line or a primary cell line which senesces after a period of passages). An initial layer that is deposited on the solid surface (or on a layer of matrix material that does not include cells that is deposited on the solid surface) includes living tumor cells obtained from the individual patient. If characterization of the tumor indicated that it is inflamed/infiltrated with immune cells this initial layer can include such immune cells (e.g. stimulatory and suppressive cells of the innate and acquired immune system, including T cells, NK cells, B cells, dendritic cells, mast cells, neutrophils, macrophages, etc.). These immune cells can be obtained from the patient. Alternatively, such immune cells can be immune cells that have been modified following collection from the patient so as to model the results of cell-directed immune therapy utilizing such modified immune cells. Alternatively, in some embodiments such an initial layer can include tumor cells and stromal cells. In some embodiments the initial layer can include tumor cells, stromal cells, and immune cells.

In some embodiments one or more additional layer(s) can be deposited over the entirety or at least a portion of the initial layer. Such an additional layer that can replicate aspects of the environment proximal to the individual's tumor (e.g., encapsulation by a layer of stromal cells). Such an additional layer can be deposited over the initial tumor-cell containing portion of the model. For example, if characterization indicates that the tumor is encapsulated in a layer that includes stromal cells, such an additional layer can include stromal cells. Suitable stromal cells include fibroblasts, endothelial cells, mesenchymal stem cells (MSC), adipocytes, and/or pericytes. In some embodiments this additional layer can include components of the extracellular matrix, biopolymer scaffolding, and/or biomaterials. In a preferred embodiment of the inventive concept cells for such an additional layer are obtained from the patient, however in other embodiments corresponding cells obtained from cell culture or a different individual can be used. In some embodiments this additional layer can include both stromal cells and immune cells.

As noted above, characterization of an individual's tumor can indicate that immune cells are present in the tumor's proximity but are not found within the tumor itself. In such instances these immune cells can often be positioned at or near a barrier of stromal or similar cells that surrounds at least a portion of the tumor. Such a barrier can be represented in a three dimensional tumor model by the additional layer described above. If characterization indicates that immune cells are present and are in this region a second additional layer can be deposited that includes such immune cells, and positioned such that the layer containing stromal cells is positioned between the tumor cell-containing layer and the immune cell-containing layer. Such immune cells are preferably obtained from the individual. Alternatively, such immune cells can be obtained from the individual and modified prior to incorporation into the three dimensional model, in order to replicate the results of cell-directed immunotherapy in the individual. Alternatively, such immune cells can be obtained from another individual and/or tissue culture.

Once a three-dimensional tumor model is generated a liquid media (such as a tissue culture media) can be introduced to cover the model and provide oxygen and nutrition. Such media can be used as a vehicle for therapeutic compounds and therapies (e.g. chemotherapy drugs, repurposed drugs from other uses, immunomodulators, gene editing enzymes, cell therapy etc.), and can be changed as needed. In some embodiments this liquid media can include cells in suspension, such as immune cells and/or stromal cells. Such immune cells in suspension can replicate (at least in part) the presence of circulating immune cells.

Once a three dimensional tumor model is generated a proposed treatment for the individual's cancer can be applied. Suitable treatment modes include, but are not limited to, chemotherapy, immunotherapy, radiotherapy, hyperthermia, and/or combinations thereof. In some embodiments two or more treatment modes can be combined. For example, two or more chemotherapy drugs can be utilized. Alternatively, two or more of chemotherapy, immunotherapy, radiotherapy, and/or hyperthermia can be combined and applied to a three dimensional tumor model. In preferred embodiments a plurality of tumor models is generated, and two or more therapeutic approaches are tested in parallel. For example, a multiwell dish or plate can be utilized, with deposition of individual three dimensional tumor models into wells of the dish or plate for treatment in parallel.

During and/or following application of the proposed treatment the tumor model can be observed or characterized in order to determine the effect of the proposed treatment. Any suitable method of observation or characterization can be utilized. For example, if deposited on a suitably transparent surface tumor cells can be observed directly with microscopy, and can permit observation of tumor cell death, penetration of formerly separated immune cells into the tumor cell-containing portion of the model, etc. Alternatively, samples can be obtained from the tumor model and characterized (for example, by immunohistochemical staining, characterization of gene expression, etc.). In still other embodiments, samples of fluid that is in contact with the three dimensional tumor model can be obtained and characterized for content of markers for inflammation, oxidative stress, apoptosis, etc. Suitable methods of characterization include high content imaging, single cell sorting (e.g. flow cytometry), proteomic analysis and/or genomic analysis to quantitate immune cell infiltration, observation of tumor cell death, and tumor microenvironment changes. Such measurements can be obtained prior to (e.g., baseline), during, and after treatment application.

Analysis of the results of such proposed treatment permits selection of one or more treatment modes that are likely to be effective in treating a specific patient's tumor. It should be appreciated that such an approach replicates not only the tumor type, but also tumor structure and/or an ongoing response of the patient's immune system to the tumor cells, thereby providing a highly predictive model. In preferred embodiments of the inventive concept one or more steps of generation of a plurality of three dimensional tumor models, application of different treatments to such tumor models, characterization of the responses of the tumor models to such treatments, analysis of the data resulting from such characterizations, and recommendation of one or more treatment protocols is provided by an automated system.

In some embodiments historical or previously collected data, data from characterization of tumor models of the inventive concept upon application of treatment protocols, protocols recommended and/or selected, and outcomes from a number of individual patients can be aggregated as a data collection in one or more databases. Such a data collection can be used as a basis for training an artificial intelligence system, which can subsequently be used to determine or recommend one or more treatment protocol(s) for an individual that is not represented in the data collection (based at least in part on data collected from an individualized three dimensional tumor model). Such an artificial intelligence system can utilize an evolutionary algorithm, and can have a neural net architecture. Such an artificial intelligence system can be provided locally, or can be accessed remotely via an appropriate user interface (e.g., a web page or application for a portable device). In some embodiments a remotely accessible artificial intelligence system can be provided as a subscription service. In some embodiments such an artificial intelligence system can be utilized to assess and recommend a treatment protocol for an individual based solely on data from characterization of the individual's tumor, without generation and utilization of an individualized three dimensional tumor model.

Another embodiment of the inventive concept utilizes multi-layer, three dimensional construct of immune cells (e.g., peripheral blood monocytes of PBMCs, T cells, NK cells, etc.) in combination with tumor cells and/or tumor associated chemoattractants and, in some embodiments, additional cell types associated with solid tumors in vitro (e.g., epithelial cells, etc.), where the layers can be planar or essentially planar. These have a variety of uses where simulation of the in vivo interaction between tumor cells and/or tumor cell products and cellular components of the human immune system are studies and/or exploited, and where accurate and convenient imaging of layers within the construct is required for accuracy and effectiveness.

Prior art methods and compositions for generating three dimensional, layered cell-containing tumor or tissue models necessarily generate concave or convex layers of varying thickness due to meniscus formation at the upper surface of fluid components prior to polymerization. In such arrangements or models the distribution of cells within a given layer is, at least in part, an artifact of this concave or convex geometry. Accurate assessment of changes in cell population and/or distribution within such concave or convex layers cannot be accurately assessed without disassembly of the layered model, as microscopic examination is necessarily limited to a designated focal plane. For example, addition of cells in suspension to such a model can result in pooling towards the center of a concave surface or the periphery of a convex surface, as well as aggregation of cells due to simple settling. In addition, if the concavity or convexity is severe enough different layers of the model can lie within the same focal plane, making microscopy impractical for accurate quantitation.

Accordingly, in some embodiments of the inventive concept a three dimensional arrangement of distinct planar layers is provided, where all or a portion of adjacent layers are in direct physical contact with each other and wherein the layers are arranged to be parallel to the focal plane of a microscope positioned for observation within or on top of the layers. Individual layers include a polymer component (which can be cross-linked) that provides a mechanically stable gel, and can include tumor cells (e.g. cells derived from an individual, cells from an established tumor cell line, etc.), cells associated with tumors in vivo (e.g. epithelial cells, stromal cells, etc.), growth factors, chemoattractant factors, chemokines, cytokines, antibodies, and/or cells of the immune system (PBMCs, T cells, etc.). In some embodiments one or more layers can include cross linked polymer components and can exclude tumor cells, growth factors, chemoattractant factors, and or cells of the immune system, essentially acting as physical barriers.

In some embodiments the composition and/or degree of crosslinking to provide a desired density, stiffness, and/or permeability (e.g., to cells, growth factors, biomolecules, chemotherapeutic agents, immunotherapeutic agents, etc.). In some embodiments cells can be provided as cells imbedded within and through a designated layer of the construct. In other embodiments cells can be deposited on an exposed surface of a layer of a construct, and in some instances can migrate into such a layer.

In some embodiments a layer of cells can be deposited between layers of a construct, such that adjacent layers both contact the layer of cells. In other embodiments a region of cells can be provided within a layer of a construct, and positioned such that the region of cells is proximal to an adjacent layer of the construct. For example, cells that are representative of those found in a capsule or region immediately surrounding a tumor (e.g., epithelial cells, stromal cells, fibroblasts, etc.) can be disposed in such a manner, so as to emulate barriers to treatment found in tumors in vivo.

In some embodiments of the inventive concept one or more planar three dimensional construct(s) can be used to evaluate the efficacy of cell-based therapies directed to cancer cells (e.g., patient-specific tumor cells, tumor cell lines, etc.). For example, immune cells (e.g., PBMCs, T cells, NK cells, macrophages, etc.) to be utilized for such therapy can be introduced into a superficial or upper layer of a planar three dimensional construct (e.g., by incorporation into the layer and/or application as a suspension to an exposed surface of the layer). Other layers of the planar three dimensional construct can include tumor/cancer cells and/or chemoattractant compounds associated with such tumor/cancer cells. Migration of the immune cells and/or an intervening layer can be observed over time to determine aggregation, migration, multiplication, and/or cytotoxic effects of such immune cells.

In some of such embodiments a layer or an interface between layers that intervenes between the tumor/cancer cells and the immune cells can include cells that are associated with the tumor in vitro (e.g., epithelial cells, stromal cells, etc.), and migration through and/or aggregation at such a layer or interface between layers can be used to determine effectiveness of the immune cells at overcoming such barriers during in vivo treatment of cancer. In some embodiments such constructs can include or be exposed to biomolecules and/or pharmaceuticals that are directed to the tumor or cancer cells. Examples include specific antibodies, immune checkpoint protein analogs (such as analogs of PD-1 and/or PDL-1), compositions for in situ gene editing, and/or chemotherapeutic compounds.

Another embodiment of the inventive concept is utilization of a planar three dimensional construct in selective clonal expansion of immune cells (e.g., PBMCs, T cells, TCM cells, NK cells). Such clonally expanded cells can, for example, be used in cell-based immunotherapy. In such embodiments immune cells can be provided in a layer that is in direct or indirect contact with a layer that includes tumor or cancer cells and/or one or more tumor associated growth factors. Such immune cells can be integrated into a layer of a planar three dimensional construct and/or can be applied as a suspension of immune cells that is applied to an exposed surface of a layer of such a construct. In some embodiments a layer of cells characteristic of tissue surrounding a tumor can be interposed between tumor cells and immune cells presented for clonal expansion. Examples of such cells include stromal cells, such as fibroblasts.

Responsive or active immune cells can be observed to divide and form an expanded responsive or active immune cell population over time. Surprisingly, Inventors have found that the time course for this can be greatly reduced relative to conventional techniques for immune cell expansion. For example, a population of immune cells can expand by at least a factor of 10 over a period of from about 7 days to about 14 days (as opposed to 6 to 8 weeks for conventional techniques). Such methods also advantageously permit effective co-culture of such immune cells with tumor cells while avoiding unwanted immunogenic responses to non-tumor antigens present in conventional basement-membrane derived products (e.g., derived from the Engelbreth-Holm-Swarm mouse tumor) used to support tumor cells which have undesirable levels of growth factors.

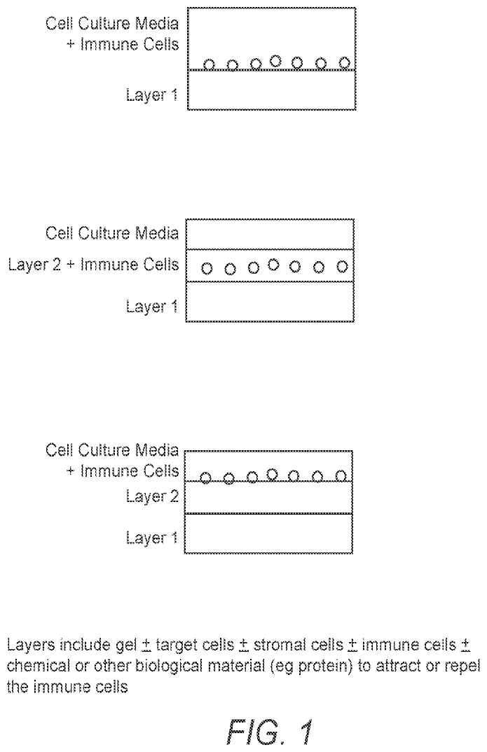

An example of a planar three dimensional construct of the inventive concept is shown below in FIG. 1 As shown, a planar three dimensional construct can include a bottom planar primary layer that includes a gel or polymer matrix. Such a matrix can be without added cells or active compounds. In some embodiments such a polymer matrix can incorporate cells or active compounds. Suitable incorporated cells include tumor or other target cells, non-tumor cells associated with a tumor (e.g., stromal cells, etc.), immune cells (e.g., T cells, NK cells, peripheral blood monocyte cells or PBMCs, etc.), active compounds (e.g. chemokines, cytokines, proteins, low molecular weight compounds) that attract immune cells, and/or active compounds that repel immune cells. Such a primary layer can be in direct contact with a liquid media. In other embodiments a planar secondary layer can be provided that is in contact with this primary layer, and can similarly include cells and/or active compounds. In such an embodiments an exposed surface of the secondary layer is in contact with liquid media. In the left panel a layer of gel or polymer that incorporates target (e.g., tumor) cells is overlaid with a layer of immune cells applied as a liquid suspension.

Examples of planar three dimensional constructs of the inventive concept are shown in FIG. 1. In the left panel a single gel or polymer layer is provided. As noted above such a polymer or gel layer can include target cells, immune cells, non-target cells that are associated with disease, and/or active compounds that influence immune cell function or activity. As shown a layer of immune cells is provided on an exposed surface of this gel layer by application of a suspension of such cells in a liquid media. The central panel shows an alternative embodiment in which a primary lower layer of gel or polymer as described above is overlaid with a secondary gel or polymer layer which can similarly contain target cells, immune cells, non-target cells, and/or active compounds that influence immune cell function or activity. In this example the secondary layer includes immune cells imbedded within the secondary layer. The right side of FIG. 1 depicts another embodiment that is similar to the embodiment shown in the central panel, however immune cells are applied to an exposed surface of the secondary layer by application of a cell suspension in liquid media. It should be appreciated that in some embodiments a layer of cells (e.g., non-tumor cells associated with a tumor, stromal cells, epithelial cells can be interposed between the primary and secondary layers, or distributed on an exposed surface of a layer; as well, the immune cells can be embedded in the 1^{st} layer and tumor cells on top of the layer or in a 2^{nd} layer.

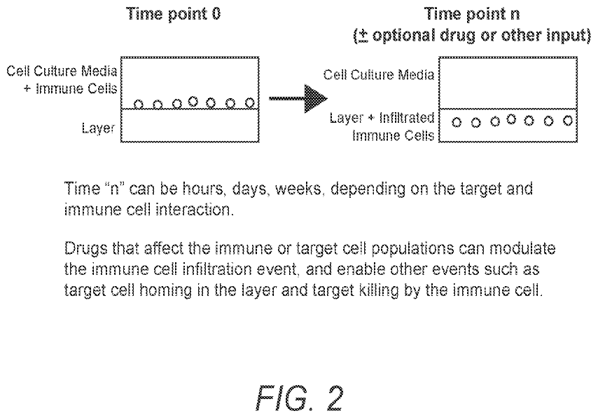

In use cells in one location of a planar three dimensional construct can migrate within the construct, for example between layers of the construct or from an exposed surface of a layer to the interior of the layer. For example, immune cells imbedded in the secondary layer of the embodiment shown in the central panel of FIG. 1 can migrate into a primary layer that includes tumor cells and/or a chemoattractant compound. Similarly, immune cells distributed on the exposed surface of a primary or secondary layer can migrate into the interior of the layer when it includes tumor cells and/or a chemoattractant compound. It should be appreciated that cells provided as a layer on top of a secondary gel or polymer layer of a construct can migrate into the secondary layer and subsequently into a primary layer. For example, immune cells applied as a liquid suspension to an exposed surface of a secondary layer can migrate into the secondary layer and subsequently into a primary layer that includes tumor cells and/or a chemoattractant compound. An example of such migration and infiltration are shown in FIG. 2. As well, it should be appreciated that immune cells can be co-embedded with tumor cells in the layer and can migrate within the layer.

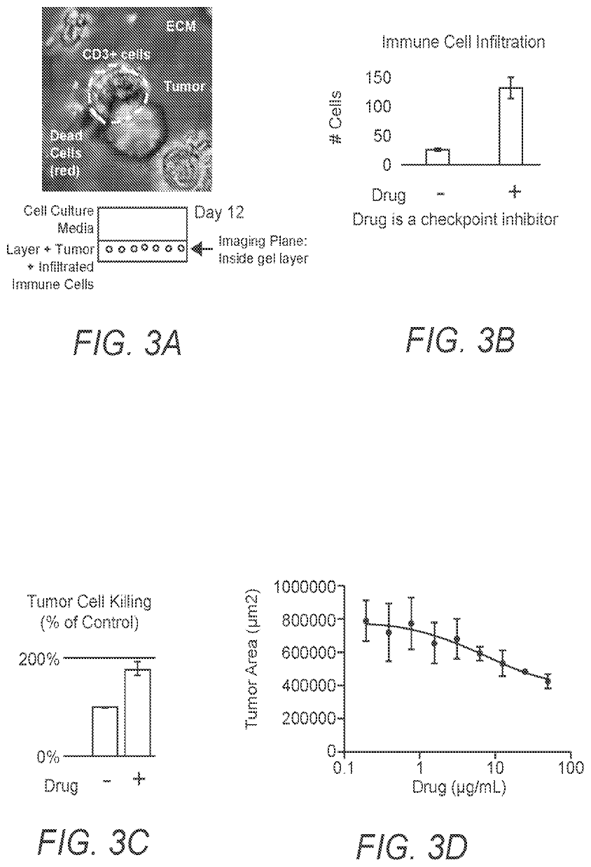

An example of immune cell migration and infiltration into a primary layer of a planar three dimensional construct that includes tumor cells is shown in FIGS. 3A to 3D. As shown in FIG. 3A, at 12 days after introduction immune cells have migrated into the primary layer to form a co-culture with tumor cells provided in the primary layer. This migration and infiltration can be enhanced by including an active compound, such as an immune checkpoint inhibitor (see FIG. 3B). Suitable checkpoint inhibitors can include drugs that block PD-1/PD-L1 and/or CTLA-4/B7-1/B7-2. Such improved infiltration of immune cells can effectively increase the effectiveness of the immune cells in killing tumor cells in the primary layer (see FIG. 3C). This is shown in FIG. 3D, which provides a histogram of typical results of such studies. As shown, tumor size decreases as the concentration of the drug (a checkpoint inhibitor) increases. It should be appreciated that such findings can be applied to assess efficacy and identify suitable checkpoint inhibitor drugs for use in an individual patient. Similarly, such findings can be applied to screen for new checkpoint inhibitor compounds.

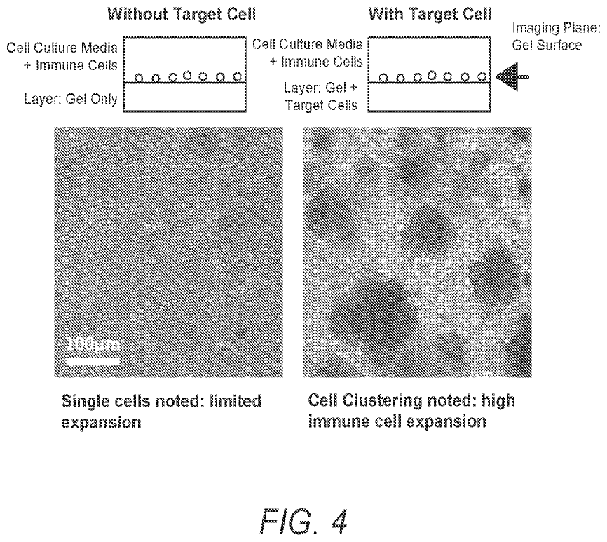

Another embodiment of the inventive concept is the use of target cells (e.g. tumor or other disease cells) imbedded in a layer of a planar three dimensional construct of the inventive concept to stimulate division and/or clonal expansion in effector cells (e.g., immune cells) at an exposed surface of the layer. Such a clonally expanded population of cells is readily harvested, for example by pipetting, manual harvesting, and/or robotic harvesting (e.g., using enzymatic or mechanical means). In some embodiments such clonal expansion occurs at an exposed surface of a gel or polymer layer that includes tumor or other disease cells. In other embodiments such clonal expansion takes place within and/or on an exposed surface of a secondary layer that is in contact with or in at least partial contact with such a primary layer of a planar three dimensional construct of the inventive concept. An example of such clonal expansion is shown in FIG. 4. As shown, in the absence of target cells in the gel/polymer layer the surface shows individual, isolated immune cells. Surprisingly, when target cells are present in the gel/polymer layer large colonies of clonally expanded immune cells develop.

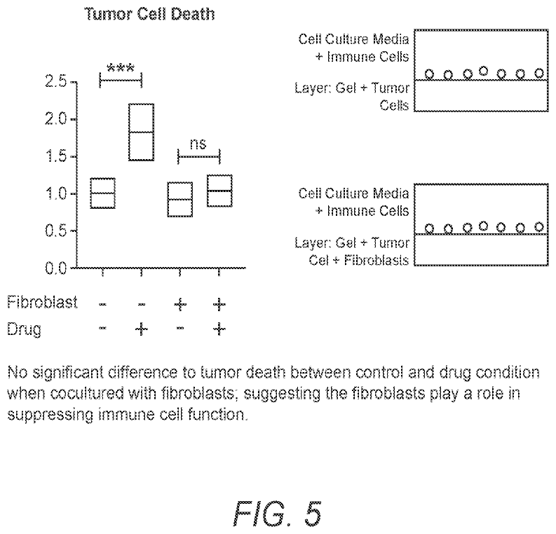

As noted above, in some embodiments non-target cells can be incorporated into one or more layers in a planar three dimensional construct of the inventive concept, at an otherwise exposed surface of such a layer, and/or at an interface between two layers of the construct. Such non-target cells can be associated with a disease state but not representative of diseased cells. For example, such a non-target cell can be a cell found in a layer of otherwise normal cells that surrounds a tumor (e.g., a stromal or epithelial cell). In some embodiments such non-target cells can interfere with migration and/or activity of effector cells (such as immune cells). As such inclusion of such non-target cells in a planar three dimensional construct of the inventive concept can render it useful as a tool to investigate methods to overcome such interference and/or to identify a patient-specific therapeutic approach. An example of such interference is shown in FIG. 5. As shown, the presence of immune cells and a checkpoint inhibitor is effective in inducing tumor cell death when the tumor cells are imbedded in a gel/polymer layer of a planar three dimensional construct of the inventive concept. This effect, however, is blocked by the inclusion of fibroblasts within the primary gel/polymer layer, at the exposed surface of the primary layer, or at an interface between primary and secondary layers.

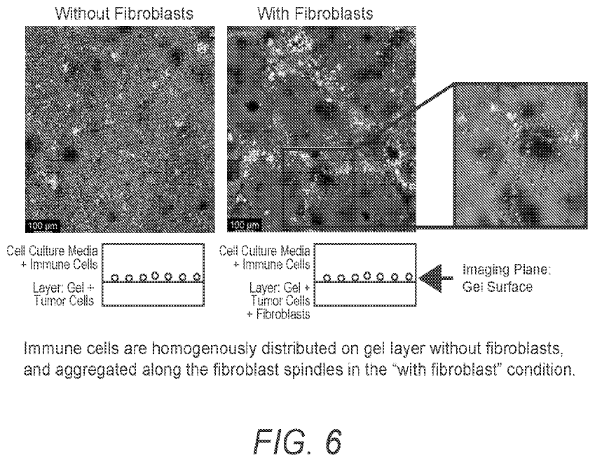

Another example of this phenomena is shown in FIG. 6, which provides a photomicrograph of the distribution of immune cells in or on a gel/polymer layer that includes tumor cells. In the left panel the gel/polymer layer does not include fibroblasts, whereas fibroblasts are included in the gel/polymer layer in the right panel. As shown, in the absence of fibroblasts immune cells are evenly distributed across the field. Surprisingly, when fibroblasts are included in the gel/polymer layer immune cells were found to aggregate along the fibroblast spindles. Without wishing to be bound by theory, the Inventor believes that such aggregation negatively impacts immune cell function and/or activity.

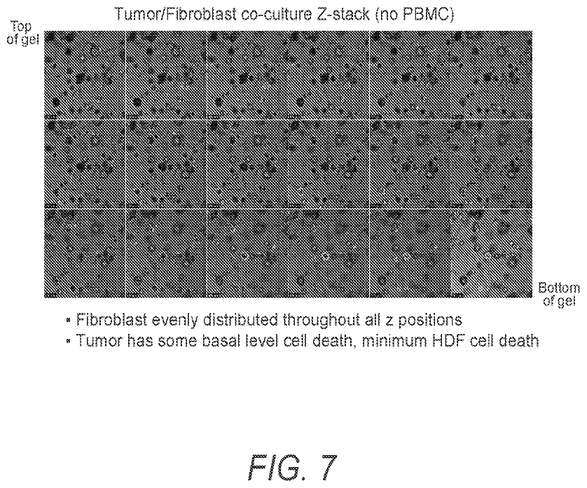

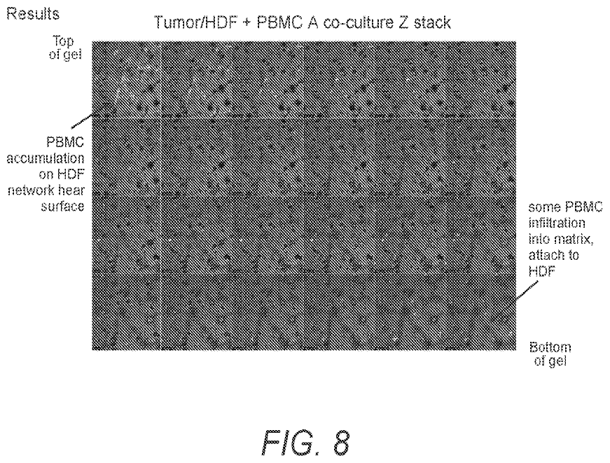

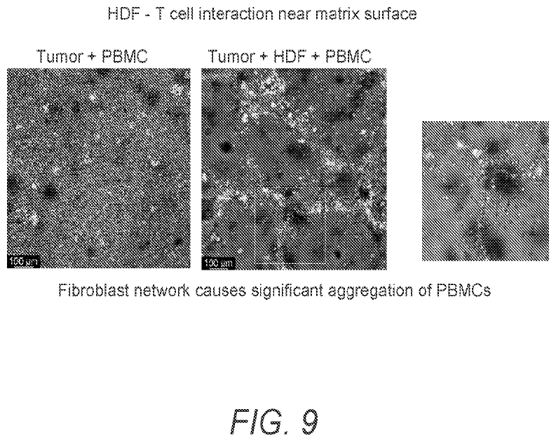

As noted above, in some embodiments planar three dimensional constructs of the inventive concept can include non-tumor cells associated with a tumor in vitro (e.g., fibroblasts, etc.). Inventors have found that planar three dimensional constructs of the inventive concept show that, in the absence of active immune cells, the presence of non-tumor cells that are associated with tumors in the construct supports a reduced death rate in tumor cells and supports formation of larger tumor cell aggregates. This effect is shown in FIG. 7. As shown in FIG. 8 when immune cells are introduced to a planar three dimensional construct of the inventive concept as shown in FIG. 7, the immune cells aggregate at the fibroblast (HDFs) present near the interface with the immune cells. This phenomena is shown in more detail in FIG. 9, which shows significant aggregation of immune cells at the fibroblast-containing interface intervening between the immune cells and the tumor cells, although some infiltration is noted. This indicates that planar three dimensional constructs of the inventive concept can be utilized as tools for investigating and circumventing insulation of tumors from cellular components of the immune system and/or cell-based immunotherapy by non-tumor cells associated with the tumor.

### FIG. 14

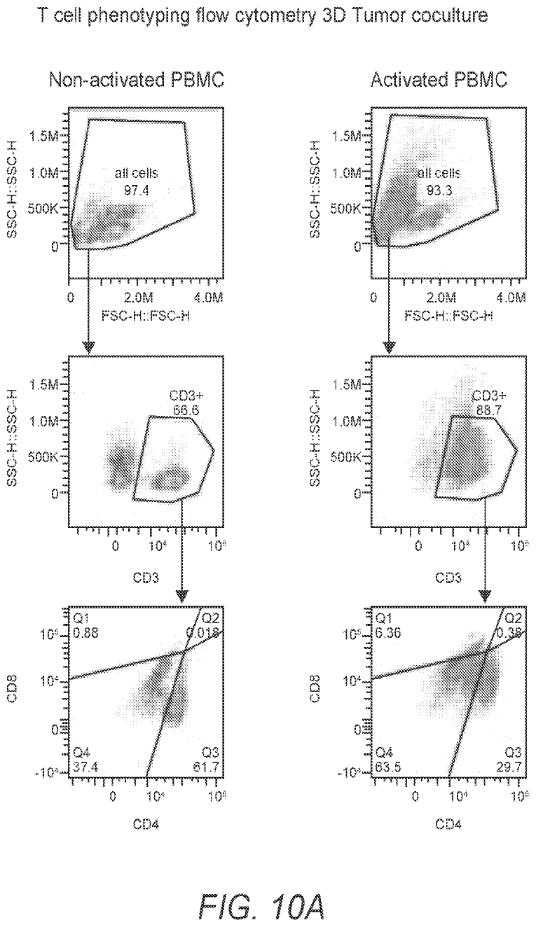

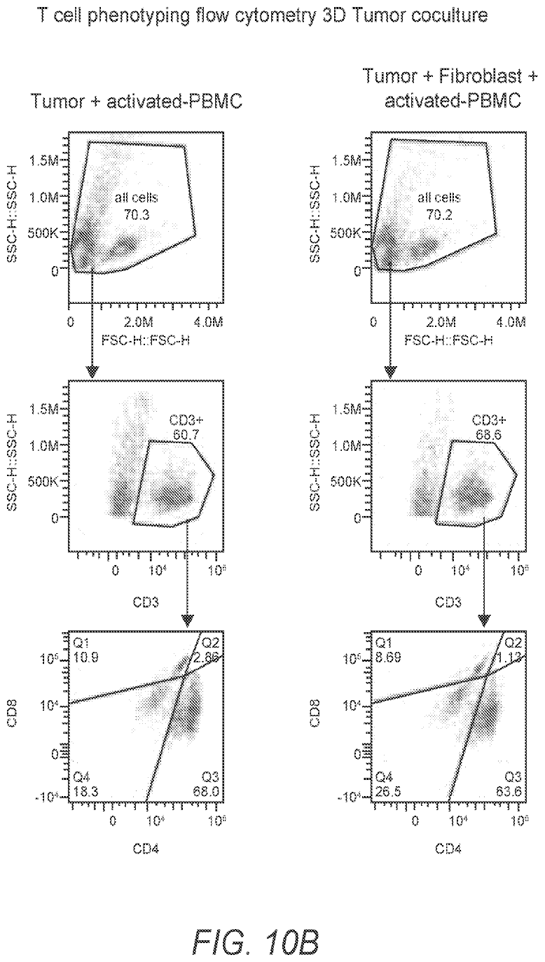

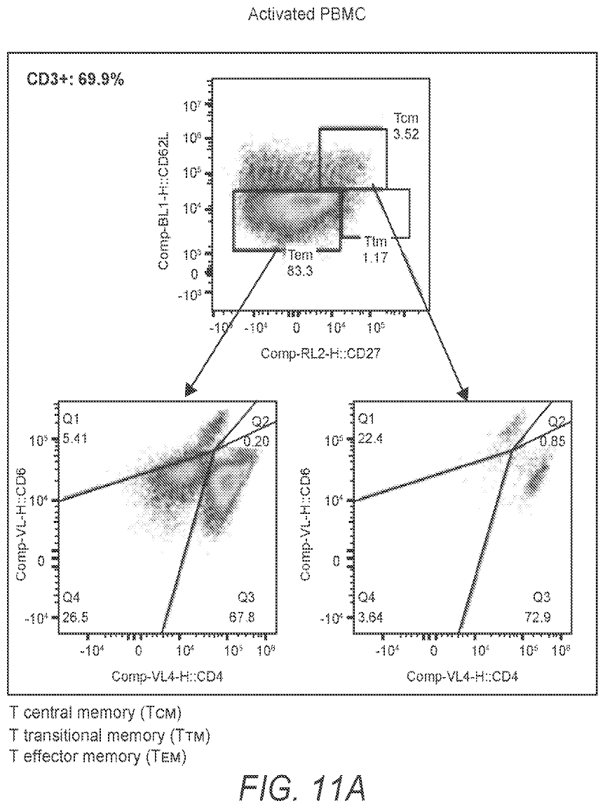

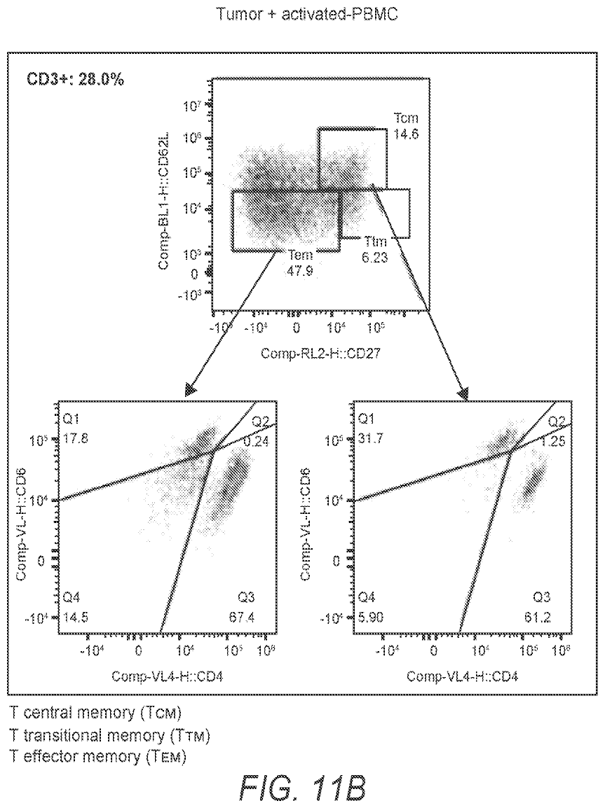

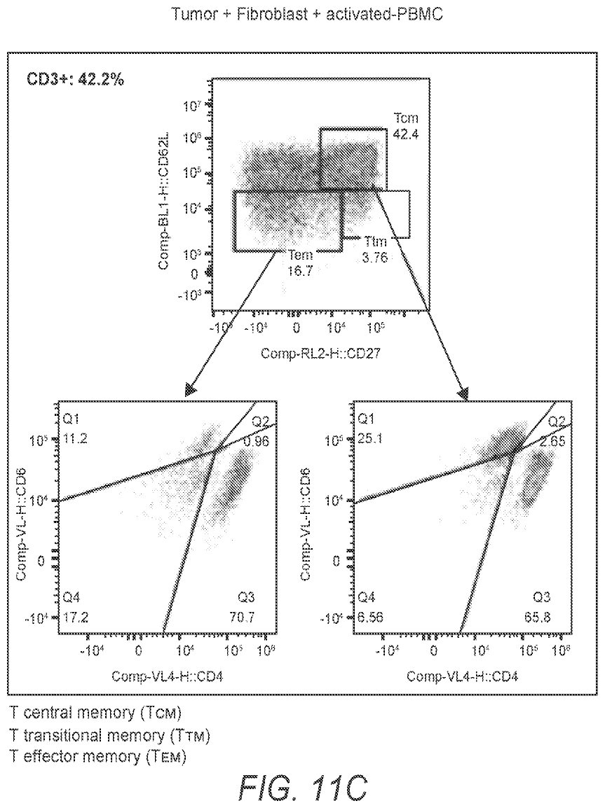

Inventors have also surprisingly found that stimulation of peripheral blood mononuclear cells (PBMCs) by tumor cells provided as three dimensional constructs as described herein can result in clonal expansion of specific immune cell phenotypes, such as memory T Cells (TCMs). Such memory T cells are highly desirable for use in immunotherapy. Inventors have also unexpectedly found that inclusion of an intervening layer of cells that can be found in tissues surrounding tumors (such as fibroblasts) positioned between the tumor cells and the PBMCs, which provides both a physical and chemical barrier, can enhance this effect. Expansion of specific T cell phenotypes from stimulated PBMCs using three dimensional tissue constructs that incorporate patient-derived tumor cells, with and without an intervening fibroblast-containing layer, is shown in FIG. 10. As shown, tumor cells and CD3+ tumor cells are evident in the co-culture. Specific T cell phenotypes can be identified by the presence or absence of characteristic cell surface markers. For example, TCM cells can be identified by the presence of certain surface markers, including CD4, CD8, CD45RA, CD45RO, CD197, CD62L, CD27, and CD95. An increase in CD4+ and CD8+ T cells is evident upon co-culturing with tumor cells. This effect is enhanced when activated PBMCs are co-cultured with three dimensional construct that provide tumor cells and an intervening layer of fibroblasts. Corresponding results are seen in similar studies performed using three dimensional tissue constructs that include patient-derived tumor cells, as shown in FIG. 11. In both studies Inventors found that use of an intervening layer that incorporates fibroblasts increased infiltrating T cells and promoted differentiation towards a specified T cell phenotype.

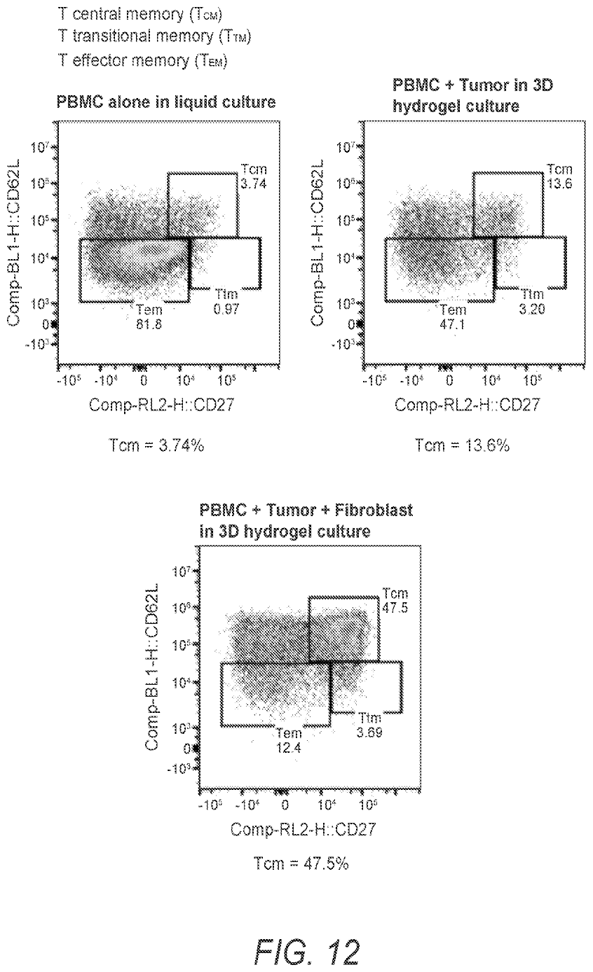

In some embodiments of the inventive concept, planar three dimensional constructs of the inventive concept can be used to induce selective clonal expansion of immune cells. As shown in FIG. 12, co-culture with tumor cells provided in a three dimensional culture of the inventive concept provides selective enhancement of clonal expansion of the TCM T cell phenotype from PBMCs, particularly when fibroblasts are provided in the three dimensional culture. Specifically, coculture of PBMCs with tumor cells provided in a three dimensional culture of the inventive concept provided an approximately 4-fold increase in clonal expansion of TCM cells relative to PBMC culture in the absence such a three dimensional culture. Co-culture of PBMCs with a three dimensional culture of the inventive concept that incorporate tumor cells and fibroblasts (as described above) provided an approximately 15-fold increase in clonal of TCM cells relative to PBMC culture in the absence such a three dimensional culture.

One embodiment of the inventive concept is a method for generating a therapeutic cell product by ex vivo stimulation of immune cells and coculture of stimulated immune with antigen-producing cells or pathogen (e.g., tumor cells or pathogen cells, or cells derived therefrom) in a three dimensional culture (e.g., one incorporating one or more tumor cell types and or cells present in encapsulating tissue that surrounds such tumors, such as fibroblasts), with subsequent expansion. The three dimensional culture can incorporates an extracellular matrix and/or a hydrogel. Specific phenotypes having therapeutic value can be selected from the resulting expanded population, for example by flow cytometry identifying one or more specific cell surface markers.

In such methods ex vivo stimulation of immune cells can be performed in any suitable fashion. Examples of suitable methods for stimulating immune cells include (but are not limited to) antigen-specific stimulation, CD3/CD28 stimulation (with or without CD137), and/or exposure to stimulating cytokines (e.g., IL2, IL7, IL15). Immune cells that are suitable for stimulation can be derived from PBMCs and/or tumor infiltrating leukocytes. Similarly, gene editing methods (e.g., CRISPR) can be applied is situ to immune cells in a planar three dimensional construct of the inventive concept in order to modulate their function. For example, one or more species of CAR-T cells can be generated in situ in such constructs in order to determine relative effectiveness (which can then be used to determine a course of treatment).

The three dimensional culture utilized in such methods can include additional components, such as cytokine-producing cells (e.g., stromal cells, fibroblasts, endothelial cells, etc.) and/or other immune cells (e.g., dendritic cells). The extracellular matrix or hydrogel component of such three dimensional cultures (e.g., dextran, gelatin, collagen, hyaluronic acid, polyethylene glycol, alginate, and/or chemically modified derivatives of these) can provide a mechanical stress component that can affect differentiation and behavior of cell in culture. Accordingly, such structural components can provide a stiffness or from about 200 Pa to about 20 kPa, or any range within this range. In some embodiments structural components of the three dimensional culture can include one or more components that are degradable by matrix metalloproteinases, which allows for antigen-producing cell (e.g., tumor cell) growth.

Three dimensional cultures utilized in such methods can include one or more layers comprising extracellular matrix and/or hydrogel components and having a thickness or depth of from about 50 μm to about 2 mm. In some embodiments thickness of a layer can vary across its width and/or length. In three dimensional cultures having multiple layers, the layers can have the same or different thicknesses. Extracellular matrix and/or hydrogel components of such three dimensional cultures can be provided as a continuous structure or as a discontinuous structure (for example, patterned into distinct areas and/or orientations). Such discontinuous or patterned cultured can provide spatial separation of distinct cell populations, and can be generated using any suitable technology (e.g., photopatterning, micropipetting, positioning of pre-formed segments, bioprinting, etc.). In some embodiments the three dimensional tissue culture can incorporate one or more porous membranes, which can be positioned within the three dimensional tissue culture (e.g., between two cell populations incorporated into the three dimensional culture) or at a surface of the three dimensional tissue culture. Such porous membranes can have a pore size ranging from about 1 μm to about 10 μm, and can enhance cell selectivity.

Immune cells provided by and/or during clonal expansion using such devices and methods can include effector T cells and memory T cells. Such memory T cells can be memory T cell stem cells, central memory T cells, effector memory T cell, and/or transitional memory T cells. Other cell populations provided by and/or during clonal expansion using such devices and methods include cytolytic effector T cells, dendritic cells, NK cells, and/or macrophages. In some embodiments an immune cell population provided by and/or during clonal expansion using such devices and methods can include a combination of two or more of these cell types.

Immune cells provided by selective clonal expansion as described above can be utilized for immunotherapy. For example, TCM cells that are derived from selective clonal expansion from PBMCs cocultured with a three dimensional culture that includes tumor cells obtained from an individual can be used to provide a patient-specific immunotherapy for that individual. In such embodiments such immune cells can be isolated (for example, using flow cytometry) and suspended in a pharmaceutically acceptable carrier. Such suspended cells can then be administered to an individual in need of treatment. For example, such immune cells can be suspended in a liquid medium and administered by intravenous infusion. Alternatively, such immune cells can be provided in a biocompatible and pharmaceutically acceptable hydrogel and/or tissue scaffold and applied locally to tissue to be treated.

As documented above, planar three dimensional constructs of the inventive concept provide for novel and useful juxtapositions between pathogenic cells (e.g. tumor cells) and components of the cellular immune system that mimic that of an infective or disease process. Such constructs can also provide for tissue or disease-specific features that can reduce the efficacy of therapeutic methods, for example the presence of a capsule or other tissue barrier around the diseased tissue. These features provide for methods of identifying or screening for effective therapeutic modes, including personalized medicine. Finally, such planar three dimensional constructs provide a mechanism for selective clonal expansion of immune cells reactive with pathogenic cells. Such expanded clones can be recovered for therapeutic use or for use in therapeutic preparations. Such findings support a wide range of systems, methods, and compositions.

Embodiments of the inventive concept include methods for diagnosing and predicting treatment for a cancer patient by obtaining data related to a pathology of a tumor of the cancer patient, determining a distribution of cancer cells, stromal cells, or immune cells within or proximal to the tumor, generating a plurality of three dimensional models of the tumor including two or more of (i) tumor cells obtained from the cancer patient, (ii) stromal cells, and/or (iii) immune cells. Cancer cells, immune cells, and/or stromal cells can be obtained from a cancer patient or from a non-patient source (i.e., an allogenic source, tissue or cell culture, explanted tissue, etc.). In such methods each of the three dimensional models reflects a distribution of cancer cells, stromal cells, or immune cells as indicated by the pathology, each of the plurality of three dimensional models is deposited on a test surface, and tumor cells of each of the three dimensional models are coupled to the test surface or an acellular layer coupled to the test surface. The plurality of three dimensional models are exposed to a plurality of anti-cancer treatments, and the effects of the anti-cancer treatments on the three dimensional tumor models are characterized. Therapeutic efficacy in the cancer patient of one or more of the anti-cancer treatments can be predicted based on their effects on the three dimensional tumor models.

Each of the plurality of three dimensional tumor models can include a first compartment and a second compartment, the first compartment having a first face and a second face, where the first face is in contact with the test surface and the second face is in contact with the second compartment, and where one or both of the first compartment and the second compartment can (optionally) include one or more of extracellular matrix, biomaterial, and a biopolymer scaffold. Tumor cells and immune cells can be co-located in this first compartment to provide a model of an immune inflamed tumor. Alternatively, tumor cells can present in and immune cells absent from the first compartment, with immune cells present in the second compartment or on a surface of the second compartment to provide a model of an immune excluded tumor. In some embodiments stromal cells are positioned proximal to the first compartment, such that stromal cells (e.g., fibroblasts, endothelial cells, mesenchymal stem cells (MSC), adipocytes, and/or pericytes) are interposed between tumor cells and immune cells (e.g., stimulatory immune cells of the innate or acquired immune system, suppressive immune cells of the innate or acquired immune system, peripheral blood mononuclear cells (PBMCs), T cells, NK cells, B cells, dendritic cells, mast cells, neutrophils, and/or macrophages). Alternatively, in some embodiments tumor cells and stromal cells can co-located in at least a portion of the plurality of three dimensional tumor models. In some embodiments tumor cells and immune cells are co-located in at least a portion of the plurality of three dimensional tumor models. In some embodiments immune cells and stromal cells are co-located in at least a portion of the plurality of three dimensional tumor models. In some embodiments tumor cells, stromal cells, and immune cells are co-located in at least a portion of the plurality of three dimensional tumor models. In some embodiments, immune cells are absent from the tumor models in order to provide a model of an immune desert tumor.

Such methods can include provision of a liquid media to a portion of the three dimensional models. The liquid media can include an immune cell and, optionally, a stromal cell. Tumor cells, stromal cells, and/or immune cells are obtained from a surgical sample of the tumor, biopsy from the tumor, normal tissue from the cancer patient, obtained from a circulating fluid of the cancer patient, or can be obtained from established cell lines. In some embodiments the immune cells are activated T cells that are prevented from infiltrating a three dimensional tumor that is under controlled conditions without treatment. Alternatively, the immune cells can activated T cells that are able to infiltrate a three dimensional tumor that is under a controlled conditions without treatment.

Data utilized in such methods can be obtained by one or more of immunohistochemistry (IHC), flow cytometry, gene expression, and methods informative of tumor microenvironment or makeup of the individual patient's tumor. IHC data so provided can be related to relative orientation and location of tumor, immune, and stromal compartments of the cancer patient. Such data can be collected by automated analysis, such as high content imaging, cell sorting, flow cytometry, proteomic analysis, expression analysis, and/or genome sequencing. In some embodiments data collected by automated analysis can be assessed to quantitate one or more of immune cell infiltration, tumor cell death, and other tumor microenvironment changes within the plurality of three dimensional tumor models

Anti-cancer treatments evaluated in such methods can be a cancer targeted therapy, an immunomodulator, a chemotherapy, a repurposed drug conventionally utilized for treatment of a condition other than cancer, radiation, or a combination of two or more of these. Three dimensional tumor models utilized in such methods can be generated using a liquid handling system, such as a bioprinter, and can be automated. Such a liquid handling system can include a photomask and a light source. Such a liquid handling system can deposit one or more of an extracellular matrix, biomaterial, or biopolymer scaffold onto the test surface.

Some embodiments of the inventive concept are methods of optimizing cancer treatment for a cancer patient by generating a first data set that includes historical predicted therapeutic efficacies developed using a method as described above for a plurality of historical cancer patients, generating a second data set that includes therapeutic outcomes for the plurality of historical cancer patients, and providing an artificial intelligence system with a learning algorithm configured to access the first and second data sets to generate a proposed treatment plan algorithm that correlates or otherwise associates predicted therapeutic efficacies and therapeutic outcomes, and applying the treatment plan algorithm to the predicted therapeutic efficacies to provide or propose one or more treatment protocols likely to be effective for treatment of the cancer patient. Such an artificial intelligence system can be configured as a neural network. In some embodiments the artificial intelligence system is accessed via an information network, and can be accessed via a subscription service.

Some embodiments of the inventive concept are methods of optimizing cancer treatment for a cancer patient by generating a first data set comprising historical predicted therapeutic efficacies using a method as described above for a plurality of historical cancer patients, generating a second data set that includes therapeutic outcomes recorded for the plurality of historical cancer patients, and providing an artificial intelligence system comprising a learning algorithm with the first and second data sets to generate a treatment plan algorithm correlating predicted therapeutic efficacies and therapeutic outcomes, and applying the treatment plan algorithm to data related to a pathology of a tumor of the cancer patient to report or propose a treatment plan for the cancer patient. Such an artificial intelligence system can be configured as a neural network. Such an artificial intelligence system can be configured as a neural network. In some embodiments the artificial intelligence system is accessed via an information network, and can be accessed via a subscription service.

Embodiments of the inventive concept include methods for performing a three-dimensional cell-based assay by obtaining a planar, three dimensional construct that includes a planar, or essentially planar, first layer of extracellular matrix or hydrogel in a culture vessel, where a first cell (e.g., immune cell, a target cells, or a combination of immune and target cells) is provided below or mixed within the first layer and a second cell (e.g., an immune cell, a target cell, and a combination of immune and target cells) is provided above the first layer, and measuring or otherwise characterizing an interaction between the first cell and the second cell interaction over a period of time. The second cell can be a tumor cell, a non-tumor diseased mammalian cell, a, pathogen, a bacteria, a virus; a source of chemokine, a source of chemoattractant, a source of a stimulus that enables an immune response, and/or an immune cell (e.g., a T cell, a B cell, a dendritic cell, a macrophage, an, NK cell, a peripheral blood mononuclear cells (PBMC), and/or derivatives of same). The second cell can be of human origin, non-human animal origin, or non-animal origin. Such a construct can further include a stromal cell, a fibroblast, an endothelial cell, an immune cell, a normal tissue cell, a diseased cell, a pathogen, a bacteria, and/or a virus.

Such a construct can include a biomaterial, such as a polymer of natural or synthetic origin; a decellularized tissue, a protein, a dextran, alginate, a poly(ethylene glycol), a collagen, a gelatin, hyaluronic acid, combinations thereof, and/or chemically modified derivatives thereof formulated for chemical or physical crosslinking. Such constructs can include at least one layer that includes a polymer that has been polymerized using electromagnetic radiation, temperature, and/or time. Such a construct can include a second layer that is planar, or essentially planar. At least one layer can be selected to be suitable for biocompatibility, elastic modulus, porosity, and degradability to enable nutrient exchange and cell interactions within a layer of a construct or between layers of a construct.

Suitable constructs for these methods can be produced using standard or custom molding methods, photolithography, bioprinting, and/or techniques that provides control of size, shape and planarity of one or more layers of the construct The first layer of such constructs can have a defined thickness and between 10 micrometers and 2 millimeters, preferably between 100 micrometers and 1 millimeter, and can have a stiffness of from 50 Pa to 20 kPa.

Embodiments of the inventive concept include a three-dimensional cell-based assay system that includes a first planar, or essentially planar, layer of extracellular matrix or hydrogel in a culture vessel, a first cell (e.g., immune cell, a target cells, or a combination of immune and target cells) below, mixed within, or above the first layer, a second layer of extracellular matrix or hydrogel on top of the first layer, and a second cell (e.g., immune cell, a target cell, a combination of immune and target cells, a tumor cell, a non-tumor diseased mammalian cell, a, pathogen, a bacteria, a virus; a source of chemokine, a source of chemoattractant, and a source of a stimulus that enables an immune response) mixed in the second layer or located on top of the second layer. Such second cells can be of human origin, non-human animal origin, or non-animal origin. Suitable immune cells include Such an assay system can further include a stromal cell, a fibroblast, an endothelial cell, an immune cell, a normal tissue cell, a diseased cell, a pathogen, a bacteria, and/or a virus. Such an assay system can include a second layer that is planar, or essentially planar. Such an assay system can include an immune cell, such as a T cell, a B cell, a dendritic cell, a macrophage, an, NK cell, a peripheral blood mononuclear cells (PBMC), and/or derivatives of these.

Such assay systems can include a biomaterial, such as a polymer of natural or synthetic origin; a decellularized tissue, a protein, a dextran, an alginate, a poly(ethylene glycol), a collagen, a gelatin, hyaluronic acid, combinations thereof, and chemically modified derivatives thereof formulated for chemical or physical crosslinking Such an assay system can include at least one layer that includes a polymer produced by polymerization using electromagnetic radiation, temperature, or time.

At least one layer of such an assay system is selected to be suitable for biocompatibility, elastic modulus, porosity, and degradability to enable nutrient exchange and cell interactions within a layer or between a plurality of layers. Such an assay system can be fabricated using standard or custom molding, photolithography, bioprinting, or a technique that provides control of size, shape and planarity of a layer of the construct. The first layer of such an assay system can have a defined thickness and between 10 micrometers and 2 millimeters, preferably between 100 micrometers and 1 millimeter, and can have a stiffness of from 50 Pa to 20 kPa.

Embodiments of the inventive concept include methods of assessing efficacy of a therapeutic modality by measuring at least one of cell viability, cell conformation, and presence of cell apoptosis markers in either of a first or second cell in a method as described above, Such measurements can be obtained by at least one of live cell image analysis, immunofluorescence, flow cytometry, proteomic analysis, and genomic analysis. Such methods can include obtaining measurements of immune cell infiltration into a layer of the construct, immune cell infiltration or into an aggregate of target cells, analysis of protein secretions, characterization of cytokines, observable changes to target cells or immune cells, characterization of apoptosis in target cells or immune cells, characterization of proliferation in target cells or immune cells, characterization of cell aggregation size in target cells or immune cells, characterization of a phenotypic change in a target cells or immune cells, and/or characterization of genotypic changes in target and immune cells. Such methods can include subjecting at least a portion of a layer of a three dimensional construct or assay system to at least one of chemical digestion, enzymatic digestion, mechanical digestion, or disruption by addition of a chelating agent. Such steps can include robotic selection or aspiration to recover a cell released by digestion or disruption of the layer.

Embodiments of the inventive concept include methods for providing individualized medicine by performing a method as described above, where the method incorporates a patient or pathogen-derived target cell and an autologous or allogenic immune cell, subjecting the construct or assay system used to one or more treatment modalities (e.g., radiotherapy, chemotherapy, targeted therapy, and/or an immunotherapy); determining efficacy of the one or more treatment modality within the construct or assay system, and providing efficacy information based on such determinations to a medical professional to assist in clinical decision-making for a specific patient or a group of patients.

Embodiments of the inventive concept include methods of expanding an immune cell population by performing a method of as described above where the three dimensional construct or assay system include a immune cells cultured proximal to a target cell within a first or second layer, providing an incubation period sufficient to allow immune cells to proliferate to generate an expanded immune cell population, and recovering the expanded immune cell population from the three dimensional construct or assay system. The expanded immune cell population is modified following recovery, for example by genetic modification, additional expansion, or a combination thereof. Such immune cells can be of autologous or allogeneic origin.

Embodiments of the inventive concept include methods of producing an antibody, by performing a method of clonal expansion as described above where the three dimensional construct or assay system includes an immune cell that is a cultured B cell or a hybridoma cell to generate a population of expanded immune cells, selecting a cell subpopulations from the population of expanded immune cells; and collecting the antibody from the selected cell subpopulation. Such selection can be performed using live imaging, manual picking, and/or robotic picking.

Embodiments of the inventive concept include methods of endogenous cell therapy by performing a method of clonal expansion as described above, where the cell to be expanded is an immune cell derived from peripheral blood, a spleen, or a disease microenvironment, expanding the immune cell to generate an expanded population of immune cells, and infusing at least a portion of the expanded population of immune cells into an individual in need of treatment. In some embodiments a subpopulation of immune cells (which can be assessed for efficacy in the patient) is separated from the expanded population of immune cells and infused into the patient in need of treatment. Assessment of patient efficacy can be performed after infusion or prior to infusion.

Embodiments of the inventive concept include methods of providing an immune cell having a specified phenotype (e.g., peripheral blood monocyte or a tumor infiltrating lymphocyte), by generating a three dimensional culture that includes an antigen producing cell (e.g. a tumor cell or a pathogen cell) layer, contacting the three dimensional culture with an immune cell to generate a coculture, and incubating the coculture for a period of time sufficient to generate the immune cell having the specified phenotype, incubating the coculture for a period of time sufficient to generate the immune cell having the specified phenotype. Suitable phenotypes include a cytolytic effector T cell, an NK cell, a macrophage, a memory T cell, such as a stem cell memory T cell, a central memory T cell, an effector memory T cell, and a transitional memory T cell. The period of time can be select to result in a stimulatory effect on the immune cell, such as antigen-specific stimulation, CD3/CD28 stimulation, CD3/CD28 stimulation with CD137, and co-stimulation by one or more cytokines (e.g., IL2, IL7, and IL15), Such a three dimensional culture can include a stromal cell layer (which can include fibroblasts) interposed between the antigen producing cell layer and the immune cell. In some embodiments the three dimensional culture can include a cytokine-producing cell (e.g., stromal cells, a fibroblast, an endothelial cell, a cytokine-producing immune cell, and/or a dendritic cell).

Such a three dimensional culture can include a hydrogel or an extracellular matrix component. The three dimensional culture can include a dextran, a gelatin, a collagen, a hyaluronic acid, and a polyethylene glycol, and at least a portion of the three dimensional culture can be degradable by a metal metalloproteinase. The three dimensional culture can have a thickness of from 20 μm to 2 mm, and can have a stiffness of from 50 Pa to 20 kPa. The three dimensional culture can be patterned into distinct areas and/or orientations, for example within a vessel or well or on a surface. In some embodiments the three dimensional culture can include a porous membrane.

Embodiments of the inventive concept include methods of providing an immunotherapy, by isolating an immune cell have a specified phenotype produced by a method as described above to provide an isolated immune cell. contacting the isolated immune cell with a pharmaceutically acceptable carrier to generate an immunotherapeutic composition, and administering the immunotherapeutic composition to an individual in need of treatment (e.g., by infusion). Suitable pharmaceutically acceptable carriers include a liquid medium that is suitable for injection or infusion. In some embodiments the pharmaceutically acceptable carrier is a biocompatible hydrogel or tissue scaffold, which can be administered by local application to a portion of the individual to be treated.

Embodiments of the inventive concept include systems for providing an immune cell having a specified phenotype, which include a three dimensional culture that include an antigen producing cell layer (e.g., a pathogenic cell layer, a tumor cell layer, etc.) and an immune cell (e.g., a peripheral blood monocyte or a tumor infiltrating lymphocyte) in coculture within the three dimensional culture, where the three dimensional culture is configured to enhance clonal expansion of the immune cell having the specified phenotype. The three dimensional culture of such a system can include a stromal cell layer that is interposed between the antigen producing cell or tumor cell layer and the immune cell. The three dimensional culture can have a defined thickness of between 10 micrometers and 2 millimeters, preferably between 100 micrometers and 1 millimeter, and can have a stiffness of from 50 Pa to 20 kPa. In such a system the three dimensional culture can patterned into distinct areas and/or orientations.

In such systems the three dimensional culture can include a stromal cell layer interposed between the antigen producing cell or tumor cell layer and the immune cell. Such a stromal cell layer can include fibroblasts. In some embodiments the three dimensional culture includes a hydrogel and/or an extracellular matrix component. At least a portion of the three dimensional culture is degradable by a metal metalloproteinase. In some embodiments the three dimensional culture can include a dextran, a gelatin, a collagen, a hyaluronic acid, an alginate, a polyethylene glycol, and/or a porous membrane.

In such systems the three dimensional culture can be configured to provide a stimulatory effect on the immune cell, such as antigen-specific stimulation, CD3/CD28 stimulation, CD3/CD28 stimulation with CD137, and co-stimulation by one or more cytokines (e.g., IL2, IL7, and/or IL15). In some embodiments the three dimensional culture includes a cytokine-producing cell. Suitable cytokine producing cells include a fibroblast, an endothelial cell, a cytokine-producing immune cell, and a dendritic cell.

In such systems the immune cell having a selected phenotype can be a memory T cell. Such a memory T cell can be a stem cell memory T cell, a central memory T cell, an effector memory T cell, and a transitional memory T cell. In some embodiments the immune cell having a selected phenotype is selected from the group consisting of one or more of a cytolytic effector T cell, an NK cell, and a macrophage.

Embodiments of the inventive concept include use of an immune cell having a specified phenotype to manufacture an immunotherapeutic composition, which includes isolating an immune cell have a specified phenotype produced by a method as described above and providing the immune cell having the specified phenotype in a pharmaceutically acceptable carrier. Such a pharmaceutically acceptable carrier can be a liquid medium suitable for infusion. Alternatively, the pharmaceutically acceptable carrier can be a biocompatible hydrogel or tissue scaffold.

Embodiments of the inventive concept include a three-dimensional cell-based assay system that has a first planar, or essentially planar, layer (which can be a product of a standard or custom molding, a photolithography, or bioprinting) of extracellular matrix or hydrogel in a culture vessel, a first cell (e.g., an immune cell, a target cell, or a combination of immune and target cells) below or mixed within the first layer, and a second layer that includes a second cell (e.g., an immune cell, a target cell, a combination of immune and target cells, a tumor cell, a non-tumor diseased mammalian cell, a, pathogen, a bacteria, a virus a source of chemokine, a source of chemoattractant, and/or a source of a stimulus that enables an immune response), where the second layer is planar. Such second cells can be of human origin, non-human animal origin, or non-animal origin. Such a system can include one or more other cell types, such as a stromal cell, a fibroblast, an endothelial cell, an immune cell, a normal tissue cell, a diseased cell, a pathogen, a bacteria, a virus, a T cell, a B cell, a dendritic cell, a macrophage, an, NK cell, a peripheral blood mononuclear cells (PBMC), and derivatives of same. In some embodiments the second layer can include extracellular matrix or hydrogel. Such systems can include a biomaterial or biocompatible material, such as a polymer of natural or synthetic origin; a decellularized tissue, a protein, a dextran, an alginate, a poly(ethylene glycol), a collagen, a gelatin, hyaluronic acid, combinations thereof, and chemically modified derivatives thereof formulated for chemical or physical crosslinking. At least one layer of such a system can include a polymer polymerized using electromagnetic radiation, temperature, or time. At least one layer of such a system can be selected to be suitable for biocompatibility, elastic modulus, porosity, and degradability to enable nutrient exchange and cell interactions within a layer or between layers In such systems the first layer can have a defined thickness and between 10 micrometers and 2 millimeters, preferably between 100 micrometers and 1 millimeter.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1-137. (canceled)

138. A method for diagnosing and predicting treatment for a cancer patient comprising:
obtaining data related to a pathology of a tumor of the cancer patient;
determining a distribution of cancer cells, stromal cells, or immune cells within or proximal to the tumor;
generating a plurality of three dimensional models of the tumor comprising two or more of tumor cells obtained from the cancer patient, stromal cells and immune cells, wherein each of the three dimensional models reflect the distribution of cancer cells, stromal cells, or immune cells as indicated by the pathology, each of the plurality of three dimensional models is deposited on a test surface, and wherein tumor cells of each of the three dimensional models are coupled to the test surface or an acellular layer coupled to the test surface;
exposing the plurality of three dimensional models to a plurality of anti-cancer treatments;
measuring effects of the anti-cancer treatments on the three dimensional tumor models; and
estimating therapeutic efficacy in the cancer patient of one or more of the anti-cancer treatments based on their effects on the three dimensional tumor models.

139. The method of claim 138, wherein each of the plurality of three dimensional tumor models comprises a first compartment and a second compartment, the first compartment having a first face and a second face, wherein the first face is in contact with the test surface and the second face is in contact with the second compartment, and wherein one or both of the first compartment and the second compartment optionally includes one or more of extracellular matrix, biomaterial, hydrogel, and biopolymer scaffold.

140. The method of claim 139, wherein tumor cells and immune cells are co-located in the first compartment to provide a model of an immune inflamed tumor.

141. The method of claim 139, wherein tumor cells are present and immune cells are absent in the first compartment, and wherein immune cells are present in the second compartment or on a surface of the second compartment to provide a model of an immune excluded tumor.

142. The method of claim 138, further comprising providing a liquid media to a portion of the three dimensional models, wherein the liquid media comprises an immune cell or a stromal cell.

143. A method of expanding an immune cell population comprising:
obtaining or generating a planar, three dimensional construct comprising a planar, or essentially planar, first layer of extracellular matrix or hydrogel in a culture vessel, wherein an immune cell is provided below or mixed within the first layer, wherein the immune cell is cultured proximal to a target cell in the first or second layer;
providing an incubation period sufficient to allow the immune cell to proliferate to generate an expanded immune cell population; and
recovering the expanded immune cell population from the construct.

144. The method of claim 143, wherein the expanded immune cell population is modified following recovery,

145. The method of claim 143, wherein the expanded immune cell population is modified by genetic modification, additional expansion, or a combination thereof.

146. The method of claim 143 wherein the immune cell is of autologous origin.

147. The method of claim 143 wherein the immune cell is of allogeneic origin.

148. A method of providing an immune cell having a specified phenotype, comprising:
generating a three dimensional culture comprising an antigen producing cell or tumor cell layer;
contacting the three dimensional culture with an immune cell to generate a coculture;
incubating the coculture for a period of time sufficient to generate the immune cell having the specified phenotype.

149. The method of claim 148, wherein the three dimensional culture comprises a stromal cell layer, wherein the stromal cell layer is interposed between or in proximity to the antigen producing cell or tumor cell layer and the immune cell.

150. The method of claim 148, wherein incubating the coculture for a period of time sufficient to generate the immune cell having the specified phenotype results in a stimulatory effect on the immune cell, wherein the stimulatory effect is selected from the group consisting of antigen-specific stimulation, CD3/CD28 stimulation, CD3/CD28 stimulation with CD137, and co-stimulation by one or more cytokines.

151. The method of claim 148, wherein the immune cell is a peripheral blood mononuclear cell or a tumor infiltrating lymphocyte.

152. The method of claim 148, wherein the three dimensional culture comprises a cytokine-producing cell.

153. The method of claim 148, wherein the immune cell having a selected phenotype is selected from the group consisting of a memory T cell, a stem cell memory T cell, a central memory T cell, an effector memory T cell, and a transitional memory T cell, a cytolytic effector T cell, an NK cell, and a macrophage.

154. The method of claim 148, wherein the three dimensional culture has a specified stiffness, wherein the specified stiffness is from 50 Pa to 20 kPa.

155. The method of claim 148, wherein the three dimensional culture has a specified thickness, wherein the specified thickness is from 20 μm to 2 mm.

156. The method of claim 148, wherein the three dimensional culture is patterned into a plurality of distinct regions or orientations.

157. The method of claim 148, wherein the three dimensional culture further comprises a porous membrane or hydrogel.
